SEQUENCE LISTING (20 sequences)

SEQ ID NO: 1 - protein, 501 aa, Homo sapiens
MQARYSVSDPNALGVVPYLSEQNYYRAAGSYGGMASPMGVYSGHPEQYSAGMGRSYAPYHHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 2 - protein, 295 aa, Mus musculus
MPRGPASLLLLVLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKTKNEDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 3 - protein, 496 aa, Mus musculus
MWQIIFLTFGWDLVLASAYSNFRKSVDSTGRRQYQVQNGPCSYTFLLPETDSCRSSSSPYMSNAVQRDAPLDYDDSVQRLQVLENILENNTQWLMKLENYIQDNMKKEMVEIQQNVVQNQTAVMIEIGTSLLNQTAAQTRKLTDVEAQVLNQTTRLELQLLQHSISTNKLEKQILDQTSEINKLQNKNSFLEQKVLDMEGKHSEQLQSMKEQKDELQVLVSKQSSVIDELEKKLVTATVNNSLLQKQQHDLMETVNSLLTMMSSPNSKSSVAIRKEEQTTFRDCAEIFKSGLTTSGIYTLTFPNSTEEIKAYCDMDVGGGGWTVIQHREDGSVDFQRTWKEYKEGFGSPLGEYWLGNEFVSQLTGQHRYVLKIQLKDWEGNEAHSLYDHFYLAGEESNYRIHLTGLTGTAGKISSISQPGSDFSTKDSDNDKCICKCSQMLSGGWWFDACGPSNLNGQYYPQKQNTNKFNGIKWYYWKGSGYSLKATTMMIRPADF

SEQ ID NO: 4 - protein, 246 aa, Mus musculus
MLGRQRIWWHLLPLLFLPFCLCQDEYMESPQAGGLPPDCSKCCHGDYGFRGYQGPPGPPGPPGIPGNHGNNGNNGATGHEGAKGEKGDKGDLGPRGERGQHGPKGEKGYPGVPPELQIAFMASLATHFSNQNSGIIFSSVETNIGNFFDVMTGRFGAPVSGVYFFTFSMMKHEDVEEVYVYLMHNGNTVFSMYSYETKGKSDTSSNHAVLKLAKGDEVWLRMGNGALHGDHQRFSTFAGFLLFETK

SEQ ID NO: 5 - protein, 348 aa, Mus musculus
MLASVAGPISLALVLLALCTRPATGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCKEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 6 - protein, 755 aa, Mus musculus
MGPTACVLVLALAILRATGQGQIPLGGDLAPQMLRELQETNAALQDVRELLRHEVKEITFLKNTVMECDACGMQPARTPGLSVRPVPLCAPGSCFPGVVCSETATGARCGPCPPGYTGNGSHCTDVNECNAHPCFPRVRCINTSPGFHCEACPPGFSGPTHEGVGLTFAKSNKQVCTDINECETGQHNCVPNSVCVNTRGSFQCGPCQPGFVGDQTSGCQRRGQHFCPDGSPSPCHEKANCVLERDGSRSCVCAVGWAGNGLLCGRDTDLDGFPDEKLRCSERQCRKDNCVTVPNSGQEDVDRDGIGDACDPDADGDGVPNEQDNCPLVRNPDQRNSDSDKWGDACDNCRSKKNDDQKDTDLDGRGDACDDDIDGDRIRNVADNCPRVPNFDQSDSDGDGVGDACDNCPQKDNPDQRDVDHDFVGDACDSDQDQDGDGHQDSRDNCPTVPNSAQQDSDHDGKGDACDDDDDNDGVPDSRDNCRLVPNPGQEDNDRDGVGDACQGDFDADKVIDKIDVCPENAEVTLTDFRAFQTVVLDPEGDAQIDPNWVVLNQGMEIVQTMNSDPGLAVGYTAFNGVDFEGTFHVNTATDDDYAGFIFGYQDSSSFYVVMWKQMEQTYWQANPFRAVAEPGIQLKAVKSSTGPGEQLRNALWHTGDTASQVRLLWKDPRNVGWKDKTSYRWFLQHRPQVGYIRVRFYEGPELVADSNVVLDTAMRGGRLGVFCFSQENIIWANLRYRCNDTIPEDYESHRLQRV

SEQ ID NO: 7 - protein, 1028 aa, Mus musculus
LAGCGHLSVSLLGLLLLLARSGTRALVCLPCDESKCEEPRSCPGSIVQGVCGCCYMCARQRNESCGGAYGLHGACDRGLRCVIRPPLNGDSITEYEVGVCEDEDWDDDQLIGFEPCNENLISGCNIINGKCECGTIRTCNNPFEFPRKDMCLSALKRIEEEKPDCSKARCEVRFSPRCPEDSILIEGYAPPGECCPLPSRCVCDPAGCLRKVCQPGYLNILVSKASGKPGECCDLYECKPVFSVDCSTVECPPVQQAVCPLDSYETQVRLTADGCCTLPARCECLSGLCGFPVCEVGSTPRIVSRGDGTPGKCCDVFECVNETKPACVFNSVEYYDGDMFRMDNCRFCRCQGGVSICFTAQCGELNCERYYVPEGECCPVCEDPIYPLNNPAGCYANGQIRAHGDRWREDDCTFCQCINGEPHCVATACGQSCMHPVKVPGECCPVCEEPTYITIDPPACGELSNCSLKEKDCVYGFKLDHNGCRTCQCKIREELCLGLKRACTLDCPFGFLTDVHNCELCQCRPRPKKCRPTMCDKFCPLGFLKNKHGCDICRCKKCPELPCSKICPLGFQQDSHGCLICKCREVPPSAGPPVLSGTCLSMDGHHHKNEESWHDGCRECYCHNGKEMCALITCPVPACGNPTIRSGQCCPSCTDDFVVQKPELSTPSICHAPGGEYFVEGETWNIDSCTQCTCHSGRVLCETEVCPPLLCQNPSRTQDSCCPQCTDDPPQPSTSHNESVPSYCRNDEGDIFLAAESWKPDACTSCVCVDSAISCYSESCPSVACERPVLRKGQCCPYCLEDTIPKKVVCHFSGKTYADEERWDIDSCTHCYCLQGQTLCSTVSCPPLPCAEPIKVEGSCCPMCPEMYVPEPTNVPIEKKNHRGEIDLEVPMWPTPSENDIIHLPRDMGHLQVDYRDNNRLHPGEDSSLDSIVSVVVPIIICLSIIIAFLLINQKKQWVPLLCWYRTPTKPSSLNNQLVSVDCKKGTRVQVDGPQRMLRIAEPDARFSGFYSMQKQNHLQADNFYQTV

SEQ ID NO: 8 - protein, 248 aa, Mus musculus
MRLLQATVLFFLLSNSLCHSEDGKDVQNDSIPTPAETSTTKASVTIPGIVSVTNPNKPADGTPPEGTTKSDVSQTSLVTTINSLTTPKHEVGTTTEGPLRNESSTMKITVPNTPTSNANSTLPGSQNKITTQLLDALPKITATPSASLTTAHTMSLLQDTEDRKIATTPSTTPSYSSIILPVVIALVVITLLVFTLVGLYRICWKRDPGTPENGNDQPQSDKESVKLLTVKTISHESGEHSAQGKTKN

SEQ ID NO: 9 - protein, 681 aa, Mus musculus
MEAAAVLPRYLQLRLLLVLLLLVLLRAGPVWPDSKVFSDLDQVRMTSEGSDCRCKCIMRPLSKDACSRVRSGRARVEDFYTVETVSSGADCRCSCTAPPSSLNPCENEWKMEKLKKQAPELLKLQSMVDLLEGALYSMDLMKVHAYIQKVASQMNTLEESIKANLSLENKVVKDSVHHLSEQLKSYENQSAIMMSIKKELSSLGLQLLQRDAAAVPATAPASSPDSKAQDTAGGQGRDLNKYGSIQKSFSDKGLAKPPKEKLLKVEKLRKESIKGRIPQPTARPRALAQQQAVIRGFTYYKAGRQEARQEARQEAPKAAADSTLKGTSWLEKLPPKIEAKLPEPNSAKHDDVRLQASEGGNLTPDITTTTTSTSSSTTTTTGTTSTTSTTSTTSTTTPSPITTPWPTEPPLHPEVPSQGREDSCEGTLRAVDPPVKHHSYGRHEGAWMKDPAALDDRIYVTNYYYGNSLVEFRNLENFKQGRWSNMYKLPYNWIGTGHVVYQGAFYYNRAFTKNIIKYDLRQRFVASWALLPDVVYEDTTPWKWRGHSDIDFAVDESGLWVIYPAVDEHDETQHEVIVLSRLDPADLSVHRETTWKTRLRRNSYGNCFLVCGILYTVDTYNQHEGQVAYAFDTHTGTDAHPQLPFLNEYSYTTQVDYNPKERVLYAWDNGHQLTYTLHFVV

SEQ ID NO: 10 - protein, 493 aa, Mus musculus
MRPLCMTYWWLGLLATVGAATGPEADVEGTEDGSQREYIYLNRYKRAGESPDKCTYTFIVPQQRVTGAICVNSKEPEVHLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKFQHLAMLAHNQSEVIAQLEEHCQRVPAARPMPQPPPAAPPRVYQPPTYNRIINQISTNEIQSDQNLKVLPPSLPTMPALTSLPSSTDKPSGPWRDCLQALEDGHSTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 11 - protein, 295 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 12 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 13 - protein, 246 aa, Homo sapiens
MLWRQLIYWQLLALFFLPFCLCQDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETK

SEQ ID NO: 14 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 15 - protein, 322 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 16 - protein, 757 aa, Homo sapiens
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRGAQRFCPDGSPSECHEHADCVLERDGSRSCVCRVGWAGNGILCGRDTDLDGFPDEKLRCPEPQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 17 - protein, 1018 aa, Homo sapiens
MYLVAGDRGLAGCGHLLVSLLGLLLLLARSGTRALVCLPCDESKEEPRNCPGSIVQGVCGCCYTCASQRNESCGGTFGIYGTCDRGLRCVIRPPLNGDSLTYEAGVCEDENWTDDQLLGFKPCNENLIAGCNIINGKCECNTIRTCSNPFEFPSQDMCSALKRIEEEKPDCSKARCEVQFSPRCPEDSVLIEGYAPPGECCPLPSRCVCNPAGCLKVCQPGNLNILVSKASGKPGECCDLYECKPVFGVDCRTVECPPVQQTACPPDSYETQRLTADGCCTLPTRCECLSGLCGFPVCEVGSTPRIVSRGDGTPGKCCDVFECVNDTKPCVFNNVEYYDGDMFRMDNCRFCRCQGGVAICFTAQCGEINCERYYVPEGECCPVCEDVYPFNNPAGCYANGLILAHGDRWREDDCTFCQCVNGERHCVATVCGQTCTNPVKVPGCCPVCEEPTIITVDPPACGELSNCTLTGKDCINGFKRDHNGCRTCQCINTEELCSERQGCTLNCPFGFLTDAQNCEICECRPRPKKCRPIICDKYCPLGLLKNKHGCDICRCKKPELSCSKICPLGFQQDSHGCLICKCREASASAGPPILSGTCLTVDGHHHKNEESWHDCRECYCLNGREMCALITCPVPACGNPTIHPGQCCPSCADDFVVQKPELSTPSICHAPGEYFVEGETWNIDSCTQCTCHSGRVLCETEVCPPLLCQNPSRTQDSCCPQCTDQPFRSLSRNNSVPNYCKNDEGDIFLAAESWKPDVCTSCICIDSVISCFSESCPSVSCERPVRKGQCCPYCIEDTIPKKVVCHFSGKAYADEERWDLDSCTHCYCLQGQTLCSTVSCPPPCVEPINVEGSCCPMCPEMYVPEPTNIPIEKTNHRGEVDLEVPLWPTPSENDIVHLPDMGHLQVDYRDNRLHPSEDSSLDSIASVVVPIIICLSIIIAFLFINQKKQWIPLLCWRTPTKPSSLNNQLVSVDCKKGTRVQVDSSQRMLRIAEPDARFSGFYSMQKQNHLQADFYQTV

SEQ ID NO: 18 - protein, 257 aa, Homo sapiens
MELLQVTILFLLPSICSSNSTGVLEAANNSLVVTTTKPSITTPNESLQKNVVTPTTGTTPKGTITNELLKMSLMSTATFLTSKDEGLKATTTDVRKNDSIINVTVTSVTLPNAVSTLQSSKPKTETQSSIKTTEIPGSVLQPDASPSKTGTLTSIPVTPENTSQSQVIGTEGGKNASTSATSRSYSSIILPVVIALIVITLSVFVLVGLYRMCWKDPGTPENGNDQPQSDKESVKLLTVKTISHESGEHSAQGKTKN

SEQ ID NO: 19 - protein, 438 aa, Homo sapiens
MSKRDKAGKDTARGKGKDISKYGSVQKSFADRGLPKPPKEKLLQVEKLRKESGKGSFLQPTAKPRALAQQQAVIRGFTYYKAGKQEVTEAVADNALQGTSWLEQLPPKVEGRSNSAEPNSAEQDEAEPRSSERVDLASGTPTSIPATTTTATTTPTPTTSLLPTEPPSGPEVSSQGREASCEGTLRAVDPPVRHHSYGRHEGAWMKDPAARDDRIYVTNYYYGNSLVEFRNLENFKQGRWSNMYKLPYNWIGTGHVVYQGAFYYNRAFTKNIIKYDLRQRFVASWALLPDVVYEDTTPWKWRGHSDIDFAVDESGLWVIYPAVDDRDEAQPEVIVLSRLDPGDLSVHRETTWKTRLRRNSYGNCFLVCGILYAVDTYNQQEGQVAYAFDTHTGTDARPQLPFLNEHAYTTQIDYNPKERVLYAWDNGHQLTYTLHFVV

SEQ ID NO: 20 - protein, 493 aa, Homo sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

(19) United States
(12) Patent Application Publication
(10) Pub. No.: US 2005/0261185 A1
(43) Pub. Date: Nov. 24, 2005

(54) Therapeutic proteins

(76) Inventors: Cecile Martijn, Sundbyberg (SE); Lena Rondahl, Bromma (SE)

Correspondence Address: FISH & RICHARDSON P.C., P.O. BOX 1022, MINNEAPOLIS, MN, 55440-1022, US

(21) Appl. No.: 11/067,121

(22) Filed: Feb. 25, 2005

Related U.S. Application Data
(60) Provisional application No. 60/576,445, filed on Jun. 2, 2004.

(30) Foreign Application Priority Data
Feb. 27, 2004 (SE) 0400489-1

(51) Int. Cl. A61K 38/17

(52) U.S. Cl. 514/12

(57) ABSTRACT

The present invention relates to a method for the treatment of a medical condition associated with obesity and/or insulin resistance, comprising administering to a subject in need thereof an effective amount of a polypeptide comprising an amino acid sequence selected from the group consisting of SEQ ID NOS:2 to 10, or a mammalian ortholog of the said polypeptide.

### CROSS REFERENCE TO RELATED APPLICATIONS

This application claims priority from Swedish Patent Application No. 0400489-1, filed Feb. 27, 2004 and U.S. Provisional Patent Application No. 60/576,445, filed Jun. 2, 2004. The prior applications are incorporated herein by reference in their entirety.

### TECHNICAL FIELD

The present invention relates to a method for the treatment of a medical condition associated with obesity and/or insulin resistance. Further, the invention relates to the use of a polypeptide in the manufacture of a medicament for the treatment of said medical condition.

### BACKGROUND

Obesity, hyperlipidemia, and insulin resistance are common forerunners of type 2 diabetes mellitus. The human winged helix/forkhead transcription factor gene foxc2 has been identified as a key regulator of adipocyte metabolism (Cederberg, A. et al. (2001) Cell 106:563-573). Increased FOXC2 expression, in adipocytes, has a pleiotropic effect on gene expression, which leads to a lean and insulin sensitive phenotype. FOXC2 affects adipocyte metabolism by increasing the sensitivity of the beta-adrenergic-cAMP-protein kinase A (PKA) signaling pathway through alteration of adipocyte PKA holoenzyme composition. Increased FOXC2 levels, induced by high fat diet, seem to counteract most of the symptoms associated with obesity, including hypertriglyceridemia and diet-induced insulin resistance; a likely consequence hereof would be protection against type 2 diabetes.

The nucleotide and amino acid sequences of the human FOXC2 protein (SEQ ID NO: 1), also known as FKHL14, FREAC-11, or S12, as well as the corresponding mouse mesenchyme forkhead-1 (MFH-1) protein, are known in the art, see Miura, N. et al. (1993) FEBS letters 326: 171-176; Miura, N. et al. (1997) Genomics 41: 489-492; WO 98/54216 and WO 01/60853.

FOXC2 is a key regulator in embryogenesis and skeletal tissue development. The expression of FOXC2 is associated with the early stage of chondrogenic differentiation both in vivo and in vitro, (i.e. related to the formation of cartilage). Bone morphogenetic proteins (BMPs) regulate FOXC2 expression in skeletal precursor cells (Nifuji A, et al. Journal of Bone and Mineral Research 2001; 16(10): 1765-1771).

The secreted frizzled-related sequence protein 2, SFRP2 (sarp1, sdf-5), is a modulator of the Wnt signaling pathway (Ladher R K, et al. Developmental Biology 2000; 218(2):183-19)—which is an important pathway for regulation of cell proliferation, differentiation, motility and morphogenesis—and an inhibitor of SFRP1 (Yoshino K, et al. Mechanisms of Development 2001; 102(1-2):45-55). SFRP2 is also involved in development and progression of cancer (Miller J R, et al. Oncogene 1999; 18(55):7860-7872). Paracrine SFRP2 induces resistance to apoptosis, i.e. programmed cell death, (Lee J L L C, et al. Autocrine/paracrine SFRP2 induces cellular resistance to apoptosis: A possible mechanism of mammary tumorigenesis. J Biol. Chem. 2004; [E.pub. ahead of print]).

Angiopoietin 2 is an antagonist of angiopoietin-1 and Tie-2 and a regulator of angiogenesis (Kim I, et al. Journal of Biological Chemistry 2000; 275(24):18550-18556; and Maisonpierre, et al. Science 1997; 277(5322):55-60). It is further reported that angiopoietin-1 possibly regulates adipose tissue growth (Dallabrida S M, et al. Biochemical and Biophysical Research Communications 2003; 311(3):563-571).

The collagenous repeat-containing sequence of 26 kDa protein, CORS26, is expressed both in cartilage (prechondrocytes), kidney and adipose tissue. It is possibly involved in skeletal development (Maeda T, et al. Journal of Biological Chemistry 2001; 276(5):3628-3634) and a possible marker for differentiation into adipocytes (Schaffler A, et al. Biochimica Et Biophysica Acta-Gene Structure and Expression 2003; 1628(1):64-70).

The connective tissue growth factor, CTGF (FISP 12, CCN2, IGFBP 8, HCS24), is induced by growth factors or certain oncogenes and binds to insulin like growth factors (IGFs) (Kim H S, et al. Proceedings of the National Academy of Sciences of the United States of America 1997; 94(24):12981-12986), and is involved in chondrocyte proliferation and angiogenesis (Nakanishi T, et al. Biochemical and Biophysical Research Communications 2001; 281(3):678-681; Perbal B. Journal of Clinical Pathology-Molecular Pathology 2001; 54(2):57-79; and Shimo T, et al. Oncology 2001; 61(4):315-322). It modulates cell signaling by bone morphogenetic proteins (BMPs) and TGFβ (Abreu J G, et al. Nature Cell Biology 2002; 4(8):599-604). CTGF combined with IGF-1 induce collagen production by high glucose leading to fibrosis and may contribute to pathogenesis of diabetic nephropathy (Lam S, et al. Diabetes 2003; 52(12):2975-2983).

Cartilage oligomeric matrix protein, COMP (thrombospondin 5), is expressed in cartilage (Hedbom E A P, et al. J Biol. Chem. 1992; 267(9):6132-6; and Fang C C C, et al. J Orthop Res. 2000; 18(4):593-603). Mutation in COMP are responsible for two chondrodysplasias, pseudoachondroplasia (PSACH) and multiple epiphyseal dysplasia (MED) (Song H R, et al. Journal of Human Genetics 2003; 48(5):222-225). COMP is regulated by BMP2 in chondrocytes (Kipnes J, et al. Osteoarthritis and Cartilage 2003; 11(6):442-454). BMP2 initiates chondrogenic lineage development (Schmitt B, et al. Differentiation 2003; 71(9-10):567-577). Further, COMP is a substrate for matrix metalloproteinases (MMPs) and ADAMs (Dickinson S C, et al. Matrix Biology 2003; 22(3):267-278). It is also expressed in blood vessels (Riessen R F M, et al. Arterioscler Thromb Vasc Biol. 2001; 21(1):47-54).

The cystein-rich motor neuron 1, CRIM1, is involved in capillary formation during angiogenesis (Glienke J, et al. Mechanisms of Development 2002; 119(2): 165-175). CRIM1 modulates the BMP activity (Wilkinson L, et al. Journal of Biological Chemistry 2003; 278(36):34181-34188).

Endomucin (gastric cancer antigen) is a vascular endothelial cell marker (Morgan S M, et al. Blood 1999; 93(1):165-175), regulated by TNFα and FGF (Liu C H, et al. Biochemical and Biophysical Research Communications 2001; 288(1):129-136). It inhibits cell adhesion and inhibition between cells and extra-cellular matrix (Kinoshita M, et al. Febs Letters 2001; 499(1-2):121-126).

Angiopoietin-like protein 2, (ANGPTL2) induce sprouting in vascular endothelial cells but does not bind to Tie-1 nor Tie-2 receptor (Kim I, et al. Journal of Biological Chemistry 1999; 274(37):26523-26528). Angiopoietin-like 3, (ANGPTL3) is reported to be secreted by liver activated lipolysis in adipocytes (Shimamura M, et al. Biochemical and Biophysical Research Communications 2003; 301(2):604-609).

### BRIEF DESCRIPTION OF THE DRAWINGS

FIG. 1 is a graph depicting the level of expression of the secreted frizzled-related sequence protein 2 in transgenic mice compared with wild-type animals.

FIG. 2 is a graph depicting the level of expression of angiopoietin 2 in transgenic mice compared to wild-type animals.

FIG. 3 is a graph depicting the level of expression of the CORS-26 in transgenic mice compared to wild-type animals.

FIG. 4 is a graph depicting the level of expression of the connective tissue growth factor (CTGF) in transgenic animals compared to their wild-type siblings.

FIG. 5 is a graph depicting the level of expression of cartilage oligomeric matrix protein (COMP) in transgenic mice compared with wild-type animals.

FIG. 6 is a graph depicting the level of expression of the cysteine-rich motor neuron 1 (CRIM 1) in transgenic mice compared with wild-type animals.

FIG. 7 is a graph depicting the level of expression of the Endomucin in transgenic mice compared with wild-type animals.

FIG. 8 is a graph depicting the level of expression of the 4932431K08Rik gene in transgenic mice compared with wild-type animals.

FIG. 9 is a graph depicting the level of expression of the angiopoietin-like 2 in transgenic mice compared to wild-type animals.

### DISCLOSURE OF THE INVENTION

It has surprisingly been shown that specific polypeptides are up-regulated in transgenic mice over-expressing FOXC2.

Consequently, in a first aspect this invention provides a method for the treatment of a medical condition associated with obesity or insulin resistance, the method comprising administering to a subject (e.g., a human) in need thereof a therapeutically effective amount of a polypeptide comprising (a) the amino acid sequence of any of SEQ ID NOS:2 to 10, or (b) a mammalian ortholog of the amino acid sequence of any of SEQ ID NOS:2 to 10. The polypeptide used in the methods described herein can optionally be formulated in a pharmaceutical composition.

The mammalian ortholog is optionally a human ortholog of the amino acid sequence of any of SEQ ID NOS:2 to 10. For example, the polypeptide used in the methods described herein can comprise, consist of, or consist essentially of the amino acid sequence of any of SEQ ID NOS:11 to 20.

In one embodiment, the invention provides a method for the treatment of a medical condition associated with obesity and/or insulin resistance, the method comprising administering to a subject (e.g., a human) in need thereof a therapeutically effective amount of a polypeptide that consists essentially of the amino acid sequence of any of SEQ ID NOS:11 to 20.

In another embodiment, the invention provides a method for the treatment of a medical condition associated with obesity and/or insulin resistance, the method comprising administering to a subject (e.g., a human) in need thereof a therapeutically effective amount of a polypeptide that consists of the amino acid sequence of any of SEQ ID NOS:11 to 20.

In a another aspect, the invention relates to the use of a polypeptide comprising an amino acid sequence selected from the group consisting of SEQ ID NOS: 2 to 10, or a mammalian ortholog of the said polypeptide, in the manufacture of a medicament for use in the treatment of a medical condition associated with obesity and/or insulin resistance.

The invention also relates to a pharmaceutical composition comprising a polypeptide comprising (a) the amino acid sequence of any of SEQ ID NOS:2 to 10, or (b) a mammalian ortholog of the amino acid sequence of any of SEQ ID NOS:2 to 10, wherein the pharmaceutical composition comprises an amount of the polypeptide effective for treating a medical condition associated with obesity or insulin resistance.

The medical condition associated with obesity and/or insulin resistance includes but is not limited to obesity, hypertriglyceridemia, diet-induced insulin resistance, and/or type 2 diabetes. The medical condition can be associated with a down-regulation of FOXC2 in the cells of the subject to be treated.

In addition to the specific amino acid sequences described herein, a polypeptide can comprise a biologically active variant or fragment (e.g., a variant or fragment that is effective in treating a medical condition associated with obesity and/or insulin resistance) of such an amino acid sequence. For example, a polypeptide used in a method or composition of the invention can comprise a fragment of an amino acid sequence described herein that retains a biological activity of the full length amino acid sequence. In another example, a polypeptide used in a method or composition of the invention can comprise a peptide sequence that is at least 80%, 85%, 90%, 95%, or 98% identical to an amino acid sequence described herein and retains a biological activity of the full length amino acid sequence.

Two molecules are said to be “homologous” if they have been derived from a common ancestor. “Orthologs” are homologs that are present within different species and have very similar or identical functions. Understanding the homology between molecules can reveal the evolutionary history of the molecules as well as information about their function; if a protein is homologous to an already characterized protein, we have a strong indication of the new protein's biochemical function.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Pharmaceutical Compositions and Methods of Administration

The pharmaceutical compositions provided herein contain therapeutically effective amounts of one or more of the polypeptides described herein that are useful in the prevention, treatment, or amelioration of one or more of the symptoms of diseases or disorders associated with obesity and/or insulin resistance, in a pharmaceutically acceptable carrier. Diseases or disorders associated with obesity and/or insulin resistance include, but are not limited to, obesity, hypertriglyceridemia, diet-induced insulin resistance, and type 2 diabetes. Pharmaceutical carriers suitable for administration of the polypeptides provided herein include any such carriers known to those skilled in the art to be suitable for the particular mode of administration.

In addition, the polypeptides may be formulated as the sole pharmaceutically active ingredient in the composition or may be combined with other active ingredients.

The compositions contain one or more polypeptides provided herein. The polypeptides are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the polypeptides described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more polypeptides is (are) mixed with a suitable pharmaceutical carrier. The concentrations of the polypeptides in the compositions are effective for delivery of an amount, upon administration, that treats, prevents, or ameliorates one or more of the symptoms of diseases or disorders associated with obesity and/or insulin resistance.

In one embodiment, the polypeptides are formulated for single dosage administration. To formulate a composition, the weight fraction of polypeptides is dissolved, suspended, dispersed or otherwise mixed in a selected carrier at an effective concentration such that the treated condition is relieved, prevented, or one or more symptoms are ameliorated.

The polypeptide is included in the pharmaceutically acceptable carrier in an amount sufficient to exert a therapeutically useful effect, preferably in the absence of undesirable side effects on the patient treated. The therapeutically effective concentration may be determined empirically by testing the polypeptides in in vitro and in vivo systems well known to those of skill in the art and then extrapolated therefrom for dosages for humans.

The concentration of polypeptide in the pharmaceutical composition will depend on absorption, inactivation and excretion rates of the polypeptide, the physicochemical characteristics of the polypeptide, the dosage schedule, and amount administered as well as other factors known to those of skill in the art. For example, the amount that is delivered is sufficient to ameliorate one or more of the symptoms of diseases or disorders associated with obesity and/or insulin resistance, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of polypeptide per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The polypeptide may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the polypeptides. The polypeptides, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the polypeptide sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing a polypeptide as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing polypeptide in the range of 0.005% to 100% with the balance made up from non-toxic carrier may be prepared. Methods for preparation of these compositions are known to those skilled in the art. The contemplated compositions may contain 0.001%-100% polypeptide, in one embodiment 0.1-95%, in another embodiment 75-85%.

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Parenteral administration of the polypeptides includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the polypeptide is adjusted so that an injection provides an effective amount to produce the desired pharmacological effect. The exact dose depends on the age, weight and condition of the patient or animal as is known in the art.

The polypeptides may be packaged as articles of manufacture containing packaging material, a polypeptide provided herein, which is effective for treatment, prevention or amelioration of one or more symptoms of obesity and/or insulin resistance, within the packaging material, and a label that indicates that the polypeptide is used for treatment, prevention or amelioration of one or more symptoms of obesity and/or insulin resistance.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the polypeptides provided herein are contemplated as are a variety of treatments for obesity and/or insulin resistance.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Below, the invention is described in the appended examples, which are intended to illustrate the invention, without limiting the scope of protection.

### EXAMPLES

Experimental Methods

Identification of genes up-regulated in the epididymal adipose tissue of transgenic mice over expressing FOXC2:

Total RNAs from epididymal fat were extracted from 3 transgenic mice (TG1, TG2 and TG3) over expressing FOXC2 (Cederberg, A. et al. (2001) Cell 106:563-573) and 3 of their wild-type siblings (WT1, WT2 and WT3). RNAs were reverse transcribed using a T7-tagged oligodT primer and double-stranded cDNAs were generated. The cDNAs were amplified using the In Vitro Transcription (IVT) with T7 RNA polymerase and biotinylated ribonucleotides. The populations of cRNAs obtained after IVT were purified with RNeasy spin columns from Qiagen and fragmented by heat to produce a distribution of RNA fragment sizes from approximately 35 to 200 bases. Affymetrix GeneChip® Expression Arrays (Murine Genome Array U74v2 sets of 3 arrays: U74Av2, U74Bv2 and U74Cv2) were hybridized (using the recommended buffer) overnight at 45° C. with the cRNA samples. The arrays were washed and stained with R-phycoerythrin streptavidin with the help of Affymetrix fluidics stations. The cartridges were scanned using Hewlett-Packard confocal scanner at 570 μm and the images were analyzed with the Affymetrix® Microarray Suite 5.0 software to identify genes differentially expressed between transgenic and wild-type mice.

### Example 1

### Secreted Frizzled-Related Sequence Protein 2

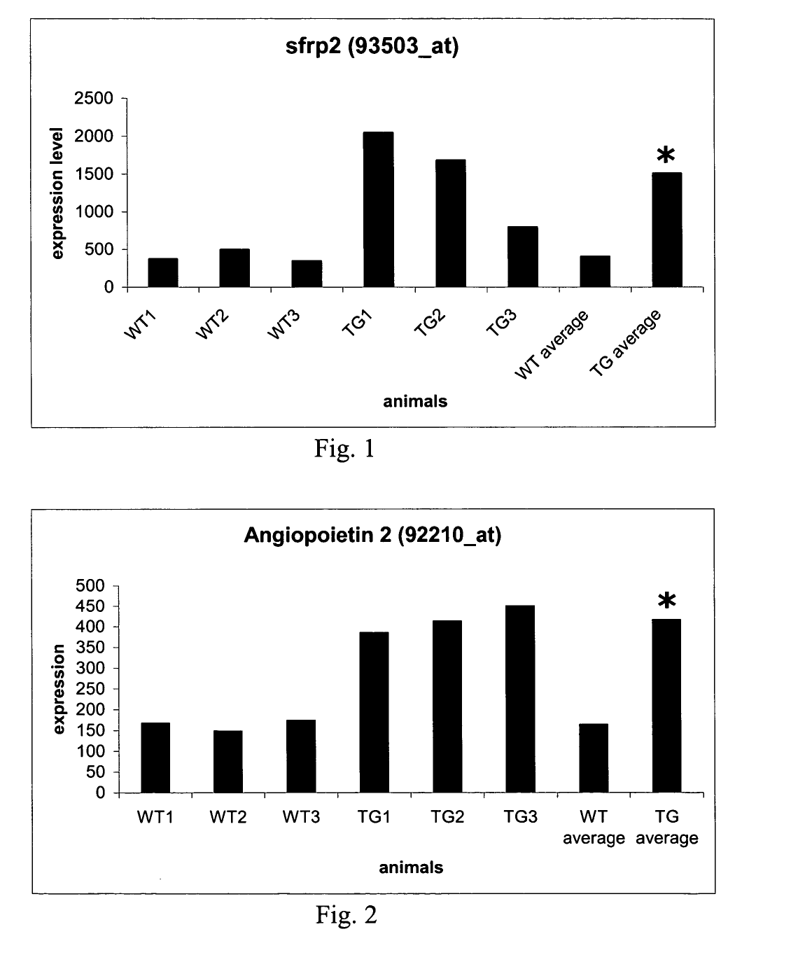

The level of expression of the secreted frizzled-related sequence protein 2 (SFRP2, Refseq ID: NM_{—}009144) was found increased 2-fold in all the transgenic animals (TG1, TG2 and TG3) compared to all their wild-type siblings (WT1, WT2 and WT3). A statistic analysis (non parametric Mann-Whitney test) performed on the average signals from the group of transgenic mice compared with the group of wild-type animals showed a significant increase (p<0.05) in expression levels in the transgenic group (FIG. 1).

### Example 2

### Angiopoietin 2

The level of expression of the angiopoietin 2 (Refseq ID: NM_{—}007426) was found significantly (p<0.05) increased in transgenic mice compared to the wild-type animals (FIG. 2).

### Example 3

### Cors-26

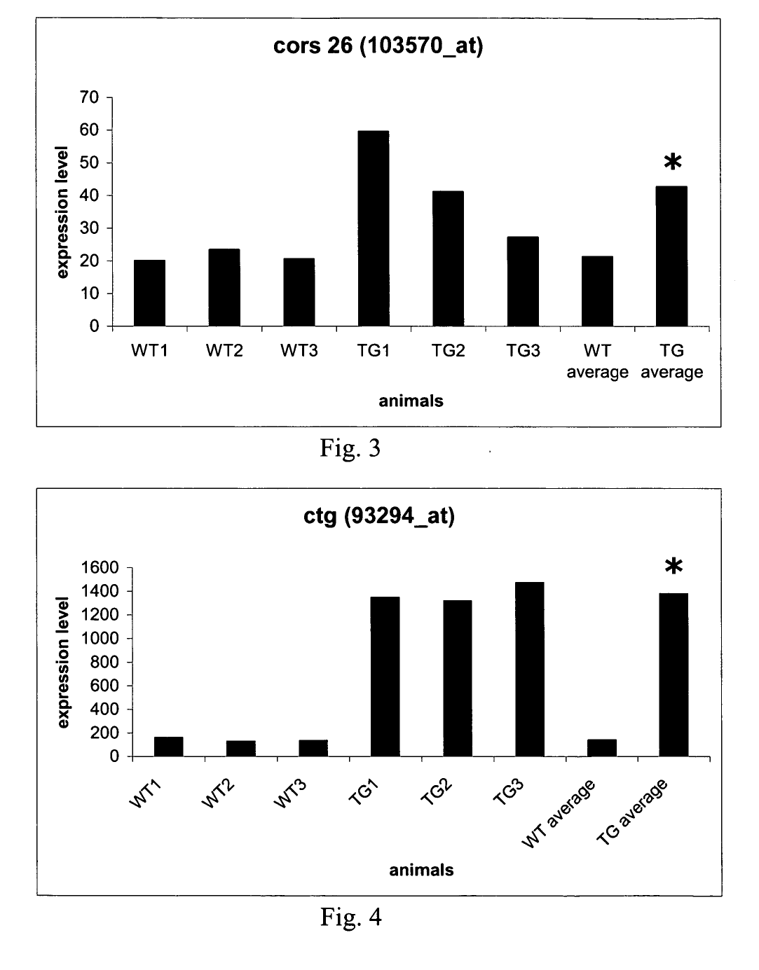

The level of expression of the Cors-26 gene (Refseq ID: NM_{—}030888) was found significantly (p<0.05) increased in transgenic mice compared to wild-type animals (FIG. 3).

### Example 4

### Connective Tissue Growth Factor

The level of expression of the Connective Tissue Growth Factor (CTGF, Refseq ID: NM_{—}010217) was found increased 6-fold in all 3 transgenic animals compared to all their wild-type siblings. A statistic analysis (non parametric Mann-Whitney test) performed on the average signals from the group of transgenic mice compared with the group of wild-type animals showed a significant increase (p<0.05) in expression levels in the transgenic group (FIG. 4).

### Example 5

### Cartilage Oligomeric Matrix Protein

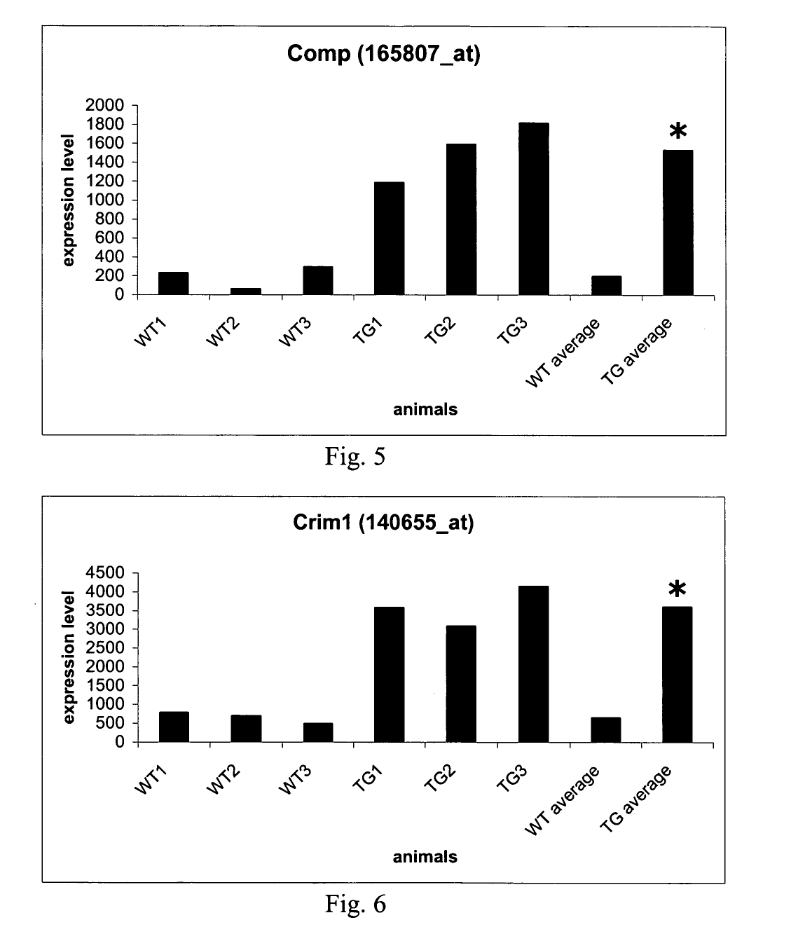

The level of expression of Cartilage Oligomeric Matrix Protein (COMP, Refseq ID: NM_{—}016685) was found increased 5-fold in all 3 transgenic animals compared to all their wild-type siblings. A statistic analysis (non parametric Mann-Whitney test) performed on the average signals from the group of transgenic mice compared with the group of wild-type animals showed a significant increase (p<0.05) in expression levels in the transgenic group (FIG. 5).

### Example 6

### Cysteine-Rich Motor Neuron 1

The level of expression of the Cysteine-Rich Motor Neuron 1 (CRIM 1, Genbank Accession No. AA990203) was found increased 4-fold in all 3 transgenic animals compared to all their wild-type siblings. A statistic analysis (non parametric Mann-Whitney test) performed on the average signals from the group of transgenic mice compared with the group of wild-type animals showed a significant increase (p<0.05) in expression levels in the transgenic group (FIG. 6).

### Example 7

### Endomucin

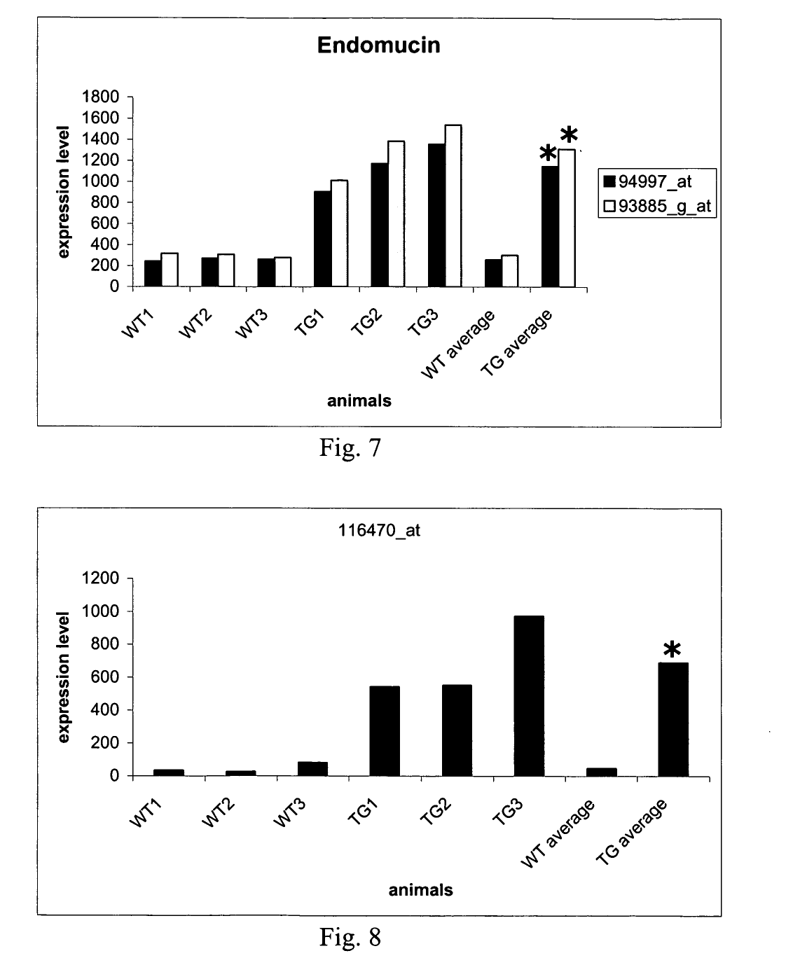

The level of expression of the Endomucin (Refseq ID: NM_{—}016885) was found increased 4-fold in all 3 transgenic animals compared to all their wild-type siblings. A statistic analysis (non parametric Mann-Whitney test) performed on the average signals from the group of transgenic mice compared with the group of wild-type animals showed a significant increase (p<0.05) in expression levels in the transgenic group (FIG. 7).

### Example 8

### 4932431K08Rik Gene

The level of expression of the 4932431K08Rik gene (Refseq ID: NM_{—}172854) was found 7-fold in all 3 transgenic animals compared to all their wild-type siblings. A statistic analysis (non parametric Mann-Whitney test) performed on the average signals from the group of transgenic mice compared with the group of wild-type animals showed a significant increase (p<0.05) in expression levels in the transgenic group (FIG. 8).

### Example 9

### Angiopoietin-Like Protein 2

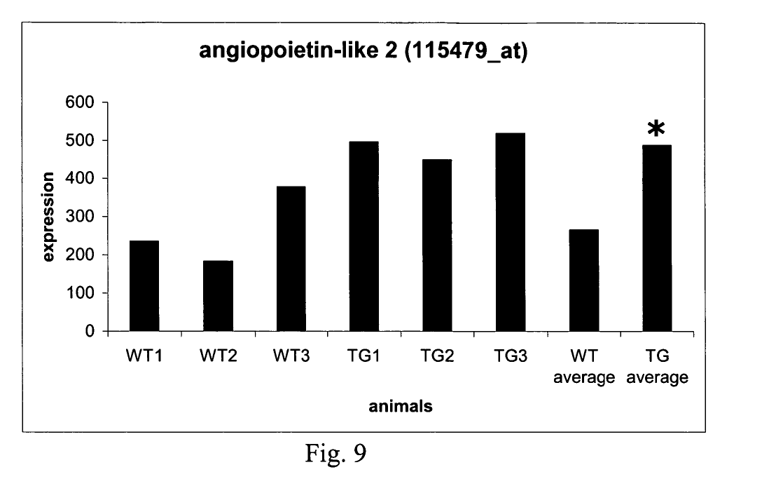

The level of expression of the angiopoietin-like 2 (Refseq ID: NM_{—}011923) was found significantly (p<0.05) increased in transgenic mice compared to the wild-type animals (FIG. 9).

### Example 10

### Reverse Transcription PCR

Quantitative PCR is performed with real-time TaqMan® and the ABI PRISM® 7900HT Sequence Detection System (Applied Biosystems). The gene of interest is quantified based on a standard curve from total RNA and normalized to the endogenous 18S ribosomal RNA gene. Total RNA is isolated from epididymal White Adipose Tissue (WAT) from at least 3 transgenic mice—overexpressing FOXC2 in WAT—and at least 3 wild-type mice. Five hundred nanograms of RNA is reverse transcribed into cDNA using the TaqMan® reverse transcription kit (Applied Biosystems). After synthesis, quantitative PCR is performed with 15 ng cDNA according to the instructions following with the TaqMan® Assays-on-Demand™ (Applied Biosystems). For the genes which are not into this collection of pre-designed primer and probe sets, a custom-designed assay is ordered (Taqman® Assays-by-Designs^{SM}, Applied Biosystems). The reactions is performed in the ABI PRISM® 7900HT and the data analyzed using the ABI PRISM® Sequence Detection System software 2.1 (Applied Biosystems).

TABLE I
Overview of sequences

| | Mouse | | | |
|---|---|---|---|---|
| | | SEQ ID | Human | |
| Name | GenBank | NO: | GenBank | SEQ ID NO: |
| SFRP-2/ | NM_009144 | 2 | AF311912 | 11 |
| SARP1 | | | | |
| Agpt2 | NM_007426 | 3 | AF004327 | 12 |
| C1q | NM_030888 | 4 | AF329837 | 13 |
| CTGF | NM_010217 | 5 | M92934 | 14 + 15 |
| | | | | two splice |
| | | | | variants |
| COMP | NM_016685 | 6 | L32137 | 16 |
| CRIM1 | AF168680 | 7 | AF167706 | 17 |
| | Partial | | | |
| Endomucin | NM_016885 | 8 | AF205940 | 18 |
| 4932431K08Rik | NM_172854 | 9 | NM_182487 | 19 |
| gene | | | | |
| Agpt-like 2 | NM_011923 | 10 | AF125175 | 20 |

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

1. A method for the treatment of a medical condition associated with obesity or insulin resistance, the method comprising administering to a subject in need thereof a therapeutically effective amount of a polypeptide comprising (a) the amino acid sequence of any of SEQ ID NOS:2 to 10, or (b) a mammalian ortholog of the amino acid sequence of any of SEQ ID NOS:2 to 10.

2. The method of claim 1, wherein the subject is a human and the polypeptide comprises a human ortholog of the amino acid sequence of any of SEQ ID NOS:2 to 10.

3. The method of claim 2, wherein the human ortholog comprises the amino acid sequence of any of SEQ ID NOS:11 to 20.

4. The method of claim 3, wherein the polypeptide comprises the amino acid sequence of SEQ ID NO:11.

5. The method of claim 3, wherein the polypeptide comprises the amino acid sequence of SEQ ID NO:12.

6. The method of claim 3, wherein the polypeptide comprises the amino acid sequence of SEQ ID NO:13.

7. The method of claim 3, wherein the polypeptide comprises the amino acid sequence of SEQ ID NO:14.

8. The method of claim 3, wherein the polypeptide comprises the amino acid sequence of SEQ ID NO:15.

9. The method of claim 3, wherein the polypeptide comprises the amino acid sequence of SEQ ID NO:16.

10. The method of claim 3, wherein the polypeptide comprises the amino acid sequence of SEQ ID NO:17.

11. The method of claim 3, wherein the polypeptide comprises the amino acid sequence of SEQ ID NO:18.

12. The method of claim 3, wherein the polypeptide comprises the amino acid sequence of SEQ ID NO:19.

13. The method of claim 3, wherein the polypeptide comprises the amino acid sequence of SEQ ID NO:20.

14. The method of claim 1, wherein the polypeptide consists essentially of the amino acid sequence of any of SEQ ID NOS:11 to 20.

15. The method of claim 1, wherein the polypeptide consists of the amino acid sequence of any of SEQ ID NOS:11 to 20.

16. The method of claim 1, wherein the medical condition is obesity, hypertriglyceridemia, diet-induced insulin resistance, or type 2 diabetes.

17. The method of claim 1, wherein the medical condition is associated with down-regulation of FOXC2.

18. A pharmaceutical composition comprising a polypeptide comprising (a) the amino acid sequence of any of SEQ ID NOS:2 to 10, or (b) a mammalian ortholog of the amino acid sequence of any of SEQ ID NOS:2 to 10, wherein the pharmaceutical composition comprises an amount of the polypeptide effective for treating a medical condition associated with obesity or insulin resistance.
